(19) United States
(12) Patent Application Publication
(10) Pub. No.: US 2010/0166685 A1
(43) Pub. Date: Jul. 1, 2010

(54) NOVEL BLOCK POLYMERS, COMPOSITIONS COMPRISING THEM, AND TREATMENT METHODS

(75) Inventor: Celine Farcet, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 12/705,239

(22) Filed: Feb. 12, 2010

Related U.S. Application Data
(62) Division of application No. 11/984,284, filed on Nov. 15, 2007.
(60) Provisional application No. 60/861,427, filed on Nov. 29, 2006.

(30) Foreign Application Priority Data
Nov. 16, 2006 (FR) 0654944

(51) Int. Cl. A61K 8/90; C08L 43/04; A61Q 17/04; A61Q 1/00; A61Q 3/00; A61Q 5/00; A61Q 19/00

(52) U.S. Cl. 424/59; 525/209; 424/78.02; 424/78.03; 424/61; 424/70.12

(57) ABSTRACT

The present disclosure relates to novel block polymers comprising particular silicone monomers and monomers of formula:

CH₂═C(R₁)—COOR₂

wherein R₁ is chosen from a hydrogen atom and a methyl group and R₂ is chosen from C₄ to C₁₂ cycloalkyl groups.

The present disclosure also relates to cosmetic or pharmaceutical compositions comprising these polymers, and also to a cosmetic treatment method using them.

This application claims benefit of U.S. Provisional Application No. 60/861,427, filed Nov. 29, 2006, the contents of which are incorporated herein by reference. This application also claims benefit of priority under 35 U.S.C. §119 to French Patent Application No. FR 06/54944, filed Nov. 16, 2006, the contents of which are also incorporated herein by reference.

The present disclosure relates to novel polymers having a specific structure and also to cosmetic compositions comprising such polymers and a cosmetic treatment method using said polymers.

Various types of polymers are conventionally used in cosmetic compositions due to the various properties that they can provide. They are, for example, used in compositions for making up or caring for the skin, lips or integuments, such as nail varnishes or compositions for the hair. However, in using two polymers that are incompatible, i.e., immiscible in the same solvent, within the same composition, the formulator may be confronted, due to the incompatibility of the polymers, with problems of phase separation, or even settling, and generally with the production of a non-homogeneous composition. Such problems could most often only be solved to date by the presence in the composition of a compound for rendering the polymers mutually compatible.

To overcome this problem, polymers of a specific structure have been proposed in European Patent Application EP 1 411 069 which comprises a first and at least a second block that are incompatible with each other and that are linked together by an intermediate block which comprises at least one monomer that is a constituent of each of said first and second blocks.

This EP application mainly describes block polymers prepared from monomers of the alkyl acrylate and methacrylate type, such as methyl, isobutyl, isobornyl or trifluoroethyl acrylate or methacrylate, or (meth)acrylic acid. The possibility of incorporating additional silicone monomers into the polymer is also mentioned generally. However, no particular advantage is mentioned in connection with this possibility.

The polymers described in the prior art may have, however, the disadvantage of being sensitive to attack by exterior fatty substances, such as for example by food oils or sebum.

The result is that the deposit may be impaired during these attacks, and become tacky. This gives the composition inferior hold properties. Furthermore, it has been observed that the comfort of the composition may also be diminished; this is because the more a deposit, or film, is sensitive to the oils, and therefore tacky, the more it will be uncomfortable to wear.

The object of the present disclosure is to provide novel polymers which may make it possible to obtain compositions that are comfortable to wear, have good hold, are not tacky, and/or are resistant to attacks by fatty substances such as oils, all while being able to be conveyed in carbon-based solvents even though they contain silicone monomers, and, in at least one embodiment, also while being shiny.

Thus, in at least one embodiment, the present disclosure relates to a block polymer comprising at least one first block and at least one second block, wherein:

the at least one first block comprises from 0.5 to 35% by weight, relative to the weight of said first block, of at least one silicone monomer chosen from, alone or as a mixture, the silicone monomers described below;

the at least one second block, identical to or different from said first block, comprises from 0.5 to 100% by weight, relative to the weight of said second block, of at least one monomer chosen from, alone or as a mixture, the monomers of formula (I): CH₂═C(R₁)—COOR₂ wherein R₁ is chosen from a hydrogen atom and a methyl group and R₂ is chosen from C₃ to C₁₂, such as C₆-C₁₀, cycloalkyl groups.

The present disclosure also relates to a composition, for example a cosmetic or pharmaceutical composition, comprising, in a physiologically acceptable medium, at least one block polymer as described above.

It has specifically been observed that the polymers according to the present disclosure may be surprisingly more comfortable than those of the prior art which do not comprise silicone monomers.

These films may also be less brittle than those of the prior art.

It has also been observed that the polymers according to the present disclosure, although they comprise silicone monomers, may have good solubility in carbon-based fatty substances, whether this is in cosmetic oils or short-chain ester type solvents, a solubility which may vary and be adjusted depending on the nature and/or the amount of monomers used. This good liposolubility may facilitate their subsequent use, such as in cosmetic compositions which generally comprise a fatty phase.

Furthermore, the polymers according to the present disclosure may make it possible to obtain films that are more or less shiny, depending on the nature and the proportion of monomers used.

Additionally, the presence of silicone monomers, even in a low amount, may make it possible to obtain polymers having a lower viscosity than that of the polymers of the prior art, which facilitates their use in cosmetic compositions.

The polymers according to the present disclosure are block polymers comprising at least one first block and at least one second block, having different glass transition temperatures (T_{g}), said first and second blocks being advantageously linked together via an intermediate segment comprising at least one constituent monomer of the first block and at least one constituent monomer of the second block.

As used herein, the terms “first” and “second” blocks do not condition the order of said blocks in the polymer structure.

The block polymer according to the present disclosure therefore comprises at least one first block and at least one second block, these blocks advantageously being incompatible with one another.

As used herein, the expression “blocks incompatible with one another” is understood to mean that the mixture formed by the polymer corresponding to the first block and by the polymer corresponding to the second block is immiscible in the predominant, by weight, polymerization solvent of the block polymer, at ambient temperature (25° C.) and atmospheric pressure (10⁵ Pa), for a content of the polymer mixture greater than or equal to 5% by weight, relative to the total weight of the mixture (polymers and solvent), it being understood that:

i) said polymers are present in the mixture in an amount such that the respective weight ratio ranges from 10/90 to 90/10; and that

ii) each of the polymers corresponding to the first and second blocks has an average (weight-average or number-average) molecular weight equal to that of the block polymer ±15%.

In the case of a mixture of polymerization solvents, assuming at least two solvents are present, said mixture of polymers is immiscible in at least one of them. Of course, in the case of a polymerization carried out in a single solvent, the latter is the predominant solvent.

The first and second blocks may, for example, be linked together by an intermediate segment comprising at least one constituent monomer m1 of the first block and at least one constituent monomer m2 of the second block.

The intermediate segment forms a block. In at least one embodiment, m2 is different from m1. The intermediate segment or block may make it possible to “compatibilize” these first and second blocks.

The block polymer of the composition according to the present disclosure is, for example, a linear, branched or grafted block ethylenic polymer, such as one that forms a deposit, and for example, one that is film-forming.

As used herein, the phrase “ethylenic polymer” is understood to mean a polymer obtained by polymerization of monomers comprising an ethylenic unsaturation.

As used herein, the phrase “block polymer” or “block copolymer” is understood to mean a polymer comprising at least two different blocks, such as at least three different blocks; for example a diblock or triblock polymer.

As used herein, the expression “polymer that forms a deposit” is understood to mean a polymer capable of forming, by itself alone or in the presence of an auxiliary agent, a deposit that adheres to a support, for example to keratinous substances.

As used herein, the phrase “film-forming polymer” is understood to mean a polymer capable of forming, by itself alone or in the presence of an auxiliary film-forming agent, a continuous film that adheres to a support, for example to keratinous substances.

Each block of the polymer according to the present disclosure is derived from one type of monomer or from several types of different monomers. This means that each block may be composed of a homopolymer or a copolymer which may be random, alternating or other.

In at least one embodiment, when it is present, the intermediate segment comprising at least one constituent monomer of the first block and at least one constituent monomer of the second block of the polymer is a random polymer. In at least another embodiment, the intermediate sequence is essentially derived from constituent monomers of the first block and of the second block.

As used herein, the term “essentially” is understood to mean at least 85%, such as at least 90%, for example at least 95%, such as 100%.

According to the present disclosure, the first and second blocks, in at least one embodiment, have different glass transition temperatures, with a difference between the glass transition temperatures of the first and second blocks that may be greater than 5° C., such as greater than 10° C. and, for example, greater than 20° C.

In at least one embodiment, the intermediate block has a glass transition temperature T_{g} between the glass transition temperatures of the first and second blocks.

The glass transition temperatures indicated are, except where mentioned otherwise, theoretical T_{g} values determined from the theoretical T_{g} values of the constituent monomers of each of the blocks, which can be found in a reference manual such as the Polymer Handbook, 4^{th} ed. (Brandrup, Immergut, Grulke), 1999, John Wiley, according to the following relationship, referred to as Fox's law:

$$\frac{1}{{T}_{g}} = {\sum }_{i} ( \frac{ω i}{{T}_{gi}} )$$

wi being the weight fraction of the monomer i in the block in question and T_{gi} being the glass transition temperature of the homopolymer of the monomer i (expressed in Kelvin).

The polymer according to the present disclosure comprises at least one first block, comprising from 0.5 to 35% by weight, relative to the weight of said first block, of at least one silicone monomer chosen from, alone or as a mixture, the following monomers:

(i) ethylenic monomers of which the ester group comprises silanes and/or siloxanes, of formula:

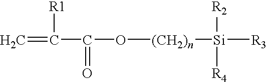

wherein:

R₁ is chosen from a hydrogen atom and a methyl group;

R₂, R₃, and R₄, which are identical or different, are chosen from C₁-C₆ alkyl groups and —OSi(R₅)₃ groups wherein R₅ is chosen from methyl and ethyl; in at least one embodiment, R₂, R₃ and/or R₄ are chosen from, independently of one another, —OSi(Me)₃ and methyl;

n is an integer ranging from 1 to 10, for example equal to 1 or 3;

(ii) polymethylsiloxane (PDMS) macromonomers, or polydimethylsiloxanes having a monoacryloyloxy or monomethacryloyloxy terminal group, of the following formula:

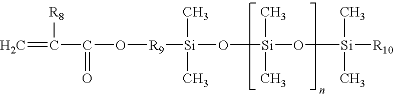

wherein:

R₈ is chosen from a hydrogen atom and a methyl group; for example methyl;

R₉ is chosen from linear and branched, for example linear, divalent hydrocarbon-based groups having from 1 to 10 carbon atoms and optionally comprising one or two ether —O— bonds; for example ethylene, propylene or butylene;

R₁₀ is chosen from linear and branched alkyl groups having from 1 to 10 carbon atoms, for example from 2 to 8 carbon atoms; for example methyl, ethyl, propyl, butyl or pentyl;

n is an integer ranging from 1 to 300, such as ranging from 3 to 200, for example ranging from 5 to 100;

(iii) ethylenic monomers of which the ester group comprises carboxysilane dendrimers of formula:

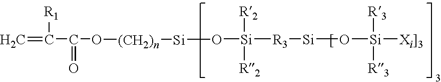

wherein:

R₁ is chosen from a hydrogen atom and a methyl group;

n is an integer ranging from 1 to 10, for example equal to 1, 2, 3 or 4;

R′₂, R″₂, R′₃ and R″₃, which are identical or different, are chosen from C₁ to C₁₀ alkyl groups, for example methyl or ethyl;

R₃ is chosen from C₂ to C₁₀, for example C₂ or C₃, alkylene divalent groups;

i is an integer ranging from 1 to 10, for example equal to 1, 2 or 3;

when i equals 2 to 10, X₍ᵢ₎), being identical or different, is chosen from —R₄—Si—[O—(R′₃)(R″₃)—X₍ᵢ₋₁₎]₃ groups wherein R₄, being identical or different, is chosen from C₂ to C₁₀, for example C₂ or C₃, alkylene divalent groups; and

when i equals 1, X₍ᵢ₎ is chosen from a hydrogen atom and C₁-C₁₀ alkyl groups, for example methyl or ethyl groups;

and for example the following monomers, wherein R₁ is chosen from a hydrogen atom and a methyl group:

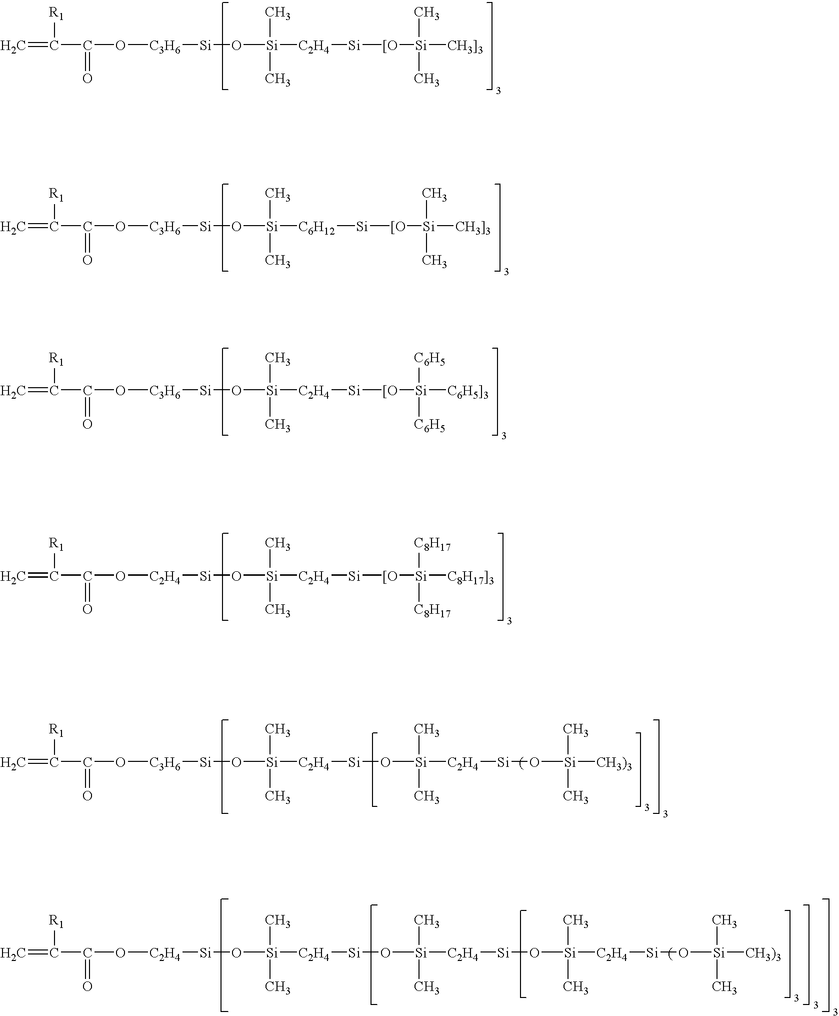

(iv) ethylenic monomers of polyhedral oligomeric silsesquioxanes (POSS) or polyhedral oligomeric silicates (POS) type of structure:

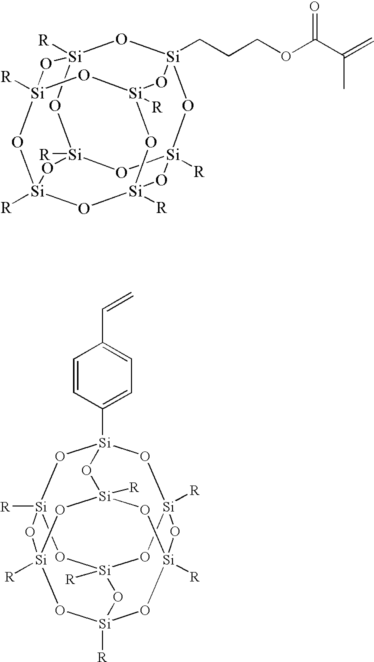

wherein R, being identical or different, is chosen from linear C₁ to C₁₀ alkyl groups, for example methyl, and cyclic C₃ to C₁₂, for example C₅, alkyl groups.

Non-limiting mention may, for example, be made of the following monomers:
(meth)acryloxypropyltris(trimethylsiloxy)silane,
(meth)acryloxypropylbis(trimethylsiloxy)methylsilane,
(meth)acryloxymethyltris(trimethylsiloxy)silane,
(meth)acryloxymethylbis(trimethylsiloxy)methylsilane;
(meth)acryloxypropyltrimethoxysilane; and, in at least one embodiment, of the following monomers:

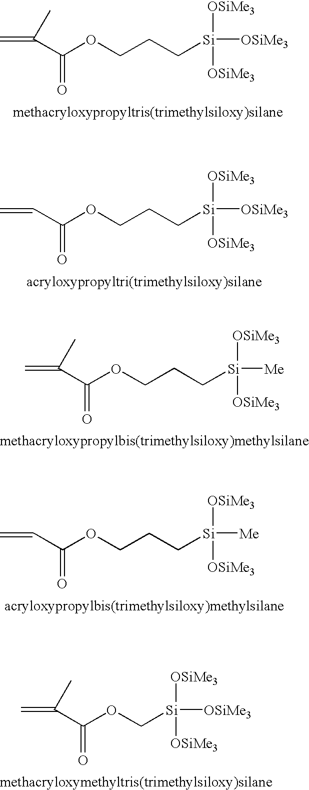

Non-limiting mention may also be made of (meth)acrylic monomers of POSS (polyhedral oligomeric silsesquioxanes) and POS (polyhedral oligomeric silicates) type, such as from Hybrid Plastics; and monomethacryloyloxypropyl polydimethylsiloxanes such as those sold under the name PS560-K6 by UCT (United Chemical Technologies Inc) or under the name MCR-M17 by Gelest Inc.

The silicone monomers described above may be present in an amount ranging from 0.5% to 35% by weight, for example from 1 to 30% by weight, such as from 1.5 to 25% by weight, for example from 2 to 20% by weight, such as from 3 to 10% by weight, of the total weight of the block comprising them.

Said block therefore further comprises from 65% to 99.5% by weight, for example from 70 to 99% by weight, such as from 75 to 98.5% by weight, for example from 80 to 98% by weight, such as from 90 to 97% by weight, relative to the total weight of said block, of at least one other monomer, for example, chosen from the additional monomers as defined below and/or the monomers of formula (I).

The polymer according to the present disclosure comprises at least one second block, being identical to or different from the first block above, comprising from 0.5 to 100% by weight, relative to the weight of said second block, of at least one monomer chosen from, alone or as a mixture, monomers of formula (I): CH₂═C(R₁)—COOR₂, wherein R₁ is chosen from a hydrogen atom and a methyl group, and R₂ is chosen from C₄ to C₁₂, for example C₆-C₁₀, cycloalkyl groups.

Thus, the silicone monomers and the monomers of formula (I) may be present in the same block or in different blocks. In at least one embodiment, they are present in different blocks.

When they are present in the same block as the silicone monomers, the monomer(s) of formula (I), in at least one embodiment, are present in an amount ranging from 0.5 to 99.5% by weight, for example from 5 to 99% by weight, such as from 20 to 90% by weight, for example from 40 to 80% by weight, such as from 50 to 70% by weight, of the total weight of the block comprising them.

When they are present in a different block from that comprising the silicone monomers, the monomer(s) of formula (I) in at least one embodiment are present in an amount ranging from 0.5 to 100% by weight, for example from 5 to 99% by weight, such as from 20 to 90% by weight, for example from 40 to 80% by weight, such as from 50 to 70% by weight, of the total weight of the block comprising them.

In at least one embodiment, the monomers of formula (I) are chosen from isobornyl methacrylate, isobornyl acrylate, cyclohexyl acrylate, cyclohexyl methacrylate, t-butylcyclohexyl acrylate, t-butylcyclohexyl methacrylate, and mixtures thereof.

In another embodiment, the block comprising the monomers of formula (I) comprises both isobornyl methacrylate and isobornyl acrylate.

In another embodiment, said block does not comprise additional monomers, and for example only comprises isobornyl methacrylate and acrylate.

In at least one embodiment, the isobornyl methacrylate is present in an amount ranging from 25% to 75% by weight, for example from 30 to 70% by weight, such as from 40 to 60% by weight, relative to the weight of the block comprising it; in at least one further embodiment, the isobornyl acrylate is present in an amount ranging from 25% to 75% by weight, for example from 30 to 70% by weight, such as from 40 to 60% by weight, relative to the weight of the block comprising it.

When the monomers of formula (I) and/or the silicone monomers are not present at 100% by weight in the block or blocks comprising them, they may comprise at least one additional monomer as defined below.

The polymer according to the present disclosure may also comprise at least one block that only comprises additional monomers; this may, for example, be the case when the monomers of formula (I) and the silicone monomers are present in the same block.

The additional monomers likely to be present in the blocks may be identical or different depending on the blocks or be within one and the same block.

They may, for example, be chosen from, alone or as a mixture, the following monomers:

(i) ethylenic hydrocarbons having from 2 to 10 carbons, such as ethylene, isoprene or butadiene;

(ii) (meth)acrylates of formula CH₂═CHCOOR′₃ or CH₂═C(CH₃)COOR′₃ wherein R′₃ is chosen from:

linear and branched alkyl groups having from 1 to 22 carbon atoms, for example from 4 to 20, such as from 6 to 18 carbon atoms, optionally inserted into which are at least one heteroatom chosen from O, N, S and P; said alkyl groups being optionally substituted by at least one substituent chosen from hydroxyl groups and halogen atoms (Cl, Br, I and F); in at least one embodiment, R′₃ may be chosen from methyl, ethyl, propyl, n-butyl, isobutyl, tert-butyl, hexyl, ethylhexyl, octyl, lauryl, isooctyl, isodecyl, dodecyl, behenyl, stearyl and ethyl-2-perfluorohexyl groups; and C₁₋₄ hydroxyalkyl groups such as 2-hydroxyethyl, 2-hydroxybutyl and 2-hydroxypropyl; and (C₁₋₄)alkoxy-(C₁₋₄)alkyl groups such as methoxyethyl, ethoxyethyl and methoxypropyl;

C₃ to C₂₀ aryl groups such as the phenyl group;

C₄ to C₃₀ aralkyl groups (C₁ to C₈ alkyl groups) such as 2-phenylethyl, t-butylbenzyl and benzyl;

heterocyclic groups having from 4 to 12 chain members comprising at least one heteroatom chosen from O, N and S, the ring being aromatic or non-aromatic;

heterocycloalkyl groups (C₁-C₄ alkyls), such as furfurylmethyl and tetrahydrofurfurylmethyl,

said aryl, aralkyl, heterocyclic and heterocycloalkyl groups being optionally substituted by at least one substituent chosen from hydroxyl groups, halogen atoms, and linear and branched C₁₋₄ alkyl groups, optionally interrupted by at least one heteroatom chosen from O, N, S and P, said alkyl groups being optionally substituted by at least one substituent chosen from hydroxyl groups and halogen atoms (Cl, Br, I and F),

groups of the formula —(C₂H₄O)ₘ—R″, wherein m is an integer ranging from 5 to 150 and R″ is chosen from a hydrogen atom and C₁ to C₃₀ alkyls, for example —POE-methyl and —POE-behenyl;

(iii) (meth)acrylamides of formula:

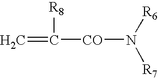

wherein R₈ is chosen from a hydrogen atom and a methyl group; and R₇ and R₆, which are identical or different, are chosen from:

hydrogen atoms; and

linear and branched alkyl groups having from 1 to 22 carbon atoms, for example from 4 to 20, such as from 6 to 18 carbon atoms, optionally interrupted by at least one heteroatom chosen from O, N, S and P; said alkyl group being optionally substituted by at least one substituent chosen from hydroxyl groups, and halogen atoms (Cl, Br, I and F); for example, R₆ and/or R₇ may be chosen from methyl, ethyl, propyl, n-butyl, isobutyl, tert-butyl, hexyl, ethylhexyl, octyl, lauryl, isooctyl, isodecyl, dodecyl, behenyl, stearyl and ethyl-2-perfluorohexyl groups; and C₁₋₄ hydroxyalkyl groups such as 2-hydroxyethyl, 2-hydroxybutyl and 2-hydroxypropyl; and (C₁₋₄)alkoxy-(C₁₋₄)alkyl groups such as methoxyethyl, ethoxyethyl and methoxypropyl;

C₃ to C₁₂ cycloalkyl groups, such as the isobornyl group, cyclohexyl and t-butylcyclohexyl;

C₃ to C₂₀ aryl groups such as the phenyl group;

C₄ to C₃₀ aralkyl groups (C₁ to C₈ alkyl groups) such as 2-phenylethyl, t-butylbenzyl and benzyl;

heterocyclic groups having from 4 to 12 chain members comprising at least one heteroatom chosen from O, N and S, the ring being aromatic or non-aromatic;

heterocycloalkyl groups (C₁-C₄ alkyl), such as furfurylmethyl and tetrahydrofurfurylmethyl,

said cycloalkyl, aryl, aralkyl, heterocyclic and heterocycloalkyl groups being optionally substituted by at least one substituent chosen from hydroxyl groups, halogen atoms, and linear and branched C₁-C₄ alkyl groups, optionally interrupted by at least one heteroatom chosen from O, N, S and P, said alkyl groups being optionally substituted by at least one substituent chosen from hydroxyl groups and halogen atoms (Cl, Br, I and F);

Non-limiting examples of (meth)acrylamide monomers are (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide, and N(2-hydroxypropylmethacrylamide).

(iv) vinyl compounds of formulae:

CH₂═CH—R₉, CH₂═CH—CH₂—R₉ or CH₂═C(CH₃)—CH₂—R₉

wherein R₉ is chosen from a hydroxyl group, Cl and F, NH₂, OR₁₄ wherein R₁₄ is chosen from a phenyl group and C₁ to C₁₂ alkyl groups (the monomer is a vinyl or allyl ether); acetamide (NHCOCH₃); an OCOR₁₅ group wherein R₁₅ is chosen from linear and branched alkyl groups having from 2 to 12 carbons (the monomer is a vinyl or allyl ester); and a group chosen from:

linear and branched alkyl groups having from 1 to 22 carbon atoms, for example from 4 to 20, such as from 6 to 18 carbon atoms, optionally interrupted by at least one heteroatom chosen from O, N, S and P; said alkyl group being optionally substituted by at least one substituent chosen from hydroxyl groups, and halogen atoms (Cl, Br, I and F);

C₃ to C₁₂ cycloalkyl groups, such as the isobornyl and cyclohexyl groups;

C₃ to C₂₀ aryl groups such as the phenyl group;

C₄ to C₃₀ aralkyl groups (C₁ to C₈ alkyl groups) such as 2-phenylethyl and benzyl;

heterocyclic groups having from 4 to 12 chain members comprising at least one heteroatom chosen from O, N and S, the ring being aromatic or non-aromatic;

heterocycloalkyl groups (C₁-C₄ alkyl groups), such as furfurylmethyl and tetrahydrofurfurylmethyl,

said cycloalkyl, aryl, aralkyl, heterocyclic and heterocycloalkyl groups being optionally substituted by at least one substituent chosen from hydroxyl groups, halogen atoms, and linear and branched C₁₋₄ alkyl groups, optionally interrupted by at least one heteroatom chosen from O, N, S and P, said alkyl groups being optionally substituted by at least one substituent chosen from hydroxyl groups and halogen atoms (Cl, Br, I and F),

Non-limiting examples of vinyl monomers are vinylcyclohexane and styrene.

Non-limiting examples of vinyl esters are vinyl acetate, vinyl propionate, vinyl butyrate, vinyl ethylhexanoate, vinyl neononanoate and vinyl neododecanoate.

Among the vinyl ethers, non-limiting mention may be made of vinyl methyl ether, vinyl ethyl ether and vinyl isobutyl ether;

(v) monomers with at least one ethylenic unsaturation comprising at least one carboxylic, phosphoric or sulphonic acid or anhydride functional group such as, for example, acrylic acid, methacrylic acid, crotonic acid, maleic anhydride, itaconic acid, fumaric acid, maleic acid, acrylamidopropanesulphonic acid, vinylbenzoic acid, vinyiphosphoric acid and the salts thereof;

(vi) monomers with at least one ethylenic unsaturation comprising at least one tertiary amine functional group such as 2-vinylpyridine, 4-vinylpyridine, dimethylaminoethyl methacrylate, diethylaminoethyl methacrylate, dimethylaminopropyl methacrylamide and the salts thereof.

The salts may be formed by neutralizing the anionic groups using a mineral base, such as LiOH, NaOH, KOH, Ca(OH)₂, NH₄OH or Zn(OH)₂; or by an organic base such as a primary, secondary or tertiary alkylamine, for example triethylamine or butylamine. This primary, secondary or tertiary alkylamine may comprise at least one nitrogen and/or oxygen atom and may therefore comprise, for example, at least one alcohol functional group; non-limiting mention may be made, for example, of 2-amino-2-methylpropanol, triethanolamine and 2-dimethylaminopropanol. Non-limiting mention may also be made of lysine and 3-(dimethylamino) propylamine.

In at least one embodiment, mention may also be made of the salts of mineral acids, such as sulphuric acid, hydrochloric acid, hydrobromic acid, hydroiodic acid, phosphoric acid and boric acid. Mention may also be made of the salts of organic acids which may comprise at least one carboxylic, sulphonic or phosphonic acid group. These may be linear, branched or cyclic aliphatic acids or aromatic acids. These acids may comprise, in addition, at least one heteroatom chosen from O and N, for example in the form of a hydroxyl group. In a further embodiment, the acids may be chosen from propionic acid, acetic acid, terephthalic acid, citric acid and tartaric acid.

When the monomers of formula (I) and the silicone monomers are present in the same block, the optional additional monomers are, for example, chosen from, alone or as a mixture:

(meth)acrylates of formula CH₂═CHCOOR′₃ or CH₂═C(CH₃)COOR′₃ wherein R′₃ is chosen from linear and branched alkyl groups, having from 1 to 22 carbon atoms, optionally interrupted by at least one heteroatom; and

monomers with at least one ethylenic unsaturation comprising at least one carboxylic acid or anhydride functional group.

For example, non-limiting mention may be made of acrylic acid, methacrylic acid and isobutyl, 2-ethylhexyl, dodecyl, stearyl and behenyl (meth)acrylates, and mixtures thereof.

When the monomers of formula (I) and the silicone monomers are not present in the same block, the optional additional monomers present in the block comprising the monomers of formula (I) and/or the optional additional monomers present in the block comprising the silicone monomers are, for example, chosen from, alone or as a mixture:

(meth)acrylates of formula CH₂═CHCOOR′₃ or CH₂═C(CH₃)COOR′₃ wherein R′₃ is chosen from linear and branched alkyl groups, having from 1 to 22 carbon atoms, optionally interrupted by at least one heteroatom; and

monomers with at least one ethylenic unsaturation comprising at least one carboxylic acid or anhydride functional group.

For example, non-limiting mention may be made of acrylic acid, methacrylic acid and isobutyl, 2-ethylhexyl, dodecyl, stearyl and behenyl (meth)acrylates, and mixtures thereof.

The additional monomers forming the optional blocks that comprise neither monomer of formula (I) nor silicone monomer may be chosen from, alone or as a mixture:

(meth)acrylates of formula CH₂═CHCOOR′₃ or CH₂═C(CH₃)COOR′₃ wherein R′₃ is chosen from linear and branched alkyl groups, having from 1 to 22 carbon atoms, optionally interrupted by at least one heteroatom; and

monomers with at least one ethylenic unsaturation comprising at least one carboxylic acid or anhydride functional group;

(iv) vinyl compounds of formulae:

CH₂—CH═R₉, CH₂═CH—CH₂—R₉ or CH₂═C(CH₃)—CH₂—R₉

wherein R₉ is chosen from a hydroxyl group, Cl and F, NH₂, OR₁₄ wherein R₁₄ is chosen from a phenyl group and C₁ to C₁₂ alkyl groups (the monomer is a vinyl or allyl ether); acetamide (NHCOCH₃); an OCOR₁₅ group wherein R₁₅ is chosen from linear and branched alkyl groups having from 2 to 12 carbons (the monomer is a vinyl or allyl ester); and a group chosen from:

linear and branched alkyl groups having from 1 to 22 carbon atoms, for example from 4 to 20, such as from 6 to 18 carbon atoms, optionally interrupted by at least one heteroatom chosen from O, N, S and P; said alkyl group being optionally substituted by at least one substituent chosen from hydroxyl groups, and halogen atoms (Cl, Br, I and F);

C₃ to C₁₂ cycloalkyl groups, such as isobornyl and cyclohexyl groups;

C₃ to C₂₀ aryl groups such as a phenyl group;

C₄ to C₃₀ aralkyl groups (C₁ to C₈ alkyl groups) such as 2-phenylethyl and benzyl;

heterocyclic groups having from 4 to 12 chain members comprising at least one heteroatom chosen from O, N and S, the ring being aromatic or non-aromatic;

heterocycloalkyl groups (C₁-C₄ alkyl groups), such as furfurylmethyl and tetrahydrofurfurylmethyl,

said cycloalkyl, aryl, aralkyl, heterocyclic and heterocycloalkyl groups being optionally substituted by at least one substituent chosen from hydroxyl groups, halogen atoms, and linear and branched C₁₋₄ alkyl groups, optionally interrupted by at least one heteroatom chosen from O, N, S and P, said alkyl groups being optionally substituted by at least one substituent chosen from hydroxyl groups and halogen atoms (Cl, Br, I and F).

For example, non-limiting mention may be made of acrylic acid, methacrylic acid and isobutyl, 2-ethylhexyl, dodecyl, stearyl and behenyl (meth)acrylates, styrene and vinyl acetate, and also mixtures thereof.

Finally, the silicone monomers, alone or as a mixture, may be present in an amount ranging from 1 to 50% by weight, for example from 2 to 40% by weight, such as from 2.5 to 35% by weight, relative to the total weight of the polymer.

The monomers of formula (I), alone or as a mixture, may be present in an amount ranging from 20 to 80% by weight, for example from 25 to 70% by weight, such as from 30 to 60% by weight, relative to the total weight of the polymer.

In at least one embodiment, the polymer according to the present disclosure comprises a block with a T_{g} greater than or equal to 20° C., for example from 20° C. to 160° C., such as from 30° C. to 140° C., for example from 40° C. to 120° C., such as from 50° C. to 110° C., and which is, in at least one embodiment, composed of carbon-based (non-silicone) monomers.

Also, in at least one embodiment, the polymer comprises a block of which the T_{g} is less than 20° C., for example from −150° C. to 20° C. exclusive of the endpoints, such as from −100° C. to 10° C., for example from −85° C. to 0° C., such as from −70° C. to −5° C., and which in at least one embodiment comprises silicone monomers.

The block having a T_{g} greater than or equal to 20° C. may for example comprise, completely or partly, monomers of which the homopolymers have a T_{g} greater than or equal to 20° C. It may also comprise monomers having a T_{g} outside of this range. These monomers and their concentration will be chosen in an appropriate manner by a person skilled in the art, for example based on Fox's law, to obtain a block with the desired T_{g}.

Among these monomers having a T_{g} greater than or equal to 20° C., non-limiting mention may be made, alone or as a mixture, of:

methacrylates of formula: CH₂═C(CH₃)—COOR₁

wherein R₁ is chosen from linear and branched unsubstituted alkyl groups comprising from 1 to 4 carbon atoms, such as methyl, ethyl, propyl and isobutyl groups; and C₄ to C₁₂ cycloalkyl groups, such as isobornyl;

acrylates of formula: CH₂═CH—COOR₂ wherein R₂ is chosen from a tert-butyl group and C₄ to C₁₂ cycloalkyl groups, such as isobornyl;

(meth)acrylamides of formula: CH₂═CR′—CO—NR₇R₈

wherein R′ is chosen from a hydrogen atom and a methyl group, and R₇ and R₈, being identical or different, are chosen from hydrogen atoms and linear and branched C₁ to C₁₂ alkyl groups such as n-butyl, t-butyl, isopropyl, isohexyl, isooctyl and isononyl groups; or R₇ is a hydrogen atom and R₈ is a 1,1-dimethyl-3-oxobutyl group; and

methacrylic acid and acrylic acid.

Among the exemplary monomers, of which the homopolymers have a glass transition temperature T_{g} greater than or equal to 20° C., non-limiting mention may be made of methyl methacrylate, ethyl methacrylate, isobutyl methacrylate, tert-butyl (meth)acrylate, (meth)acrylic acid, isobornyl (meth)acrylate, N-butylacrylamide, N-t-butylacrylamide, N-isopropylacrylamide, N,N-dimethylacrylamide, N,N-dibutylacrylamide and mixtures thereof.

The block having a T_{g} less than 20° C. may, in at least one embodiment, comprise, completely or partly, monomers of which the homopolymers have a T_{g} less than 20° C. It may also comprise monomers having a T_{g} outside of this range. These monomers and their concentration will be chosen in a suitable manner by a person skilled in the art, for example based on Fox's law, to obtain a block with the desired T_{g}.

Among these monomers having a T_{g} less than 20° C., non-limiting mention may be made, alone or as a mixture, of:

acrylates of formula CH₂═CHCOOR₃,

wherein R₃ is chosen from linear and branched C₁ to C₁₂ unsubstituted alkyl groups, except for the tert-butyl group, optionally inserted into which is at least one heteroatom chosen from O, N, and S;

methacrylates of formula CH₂═C(CH₃)—COOR₄,

wherein R₄ is chosen from linear and branched C₆ to C₁₂ unsubstituted alkyl groups, optionally interrupted by at least one heteroatom chosen from O, N and S;

vinyl esters of formula R₅—CO—O—CH═CH₂ wherein R₅ is chosen from linear and branched C₄ to C₁₂ alkyl groups;

(C₄-C₁₂ alkyl) vinyl ethers, such as methyl vinyl ether and ethyl vinyl ether;

N—(C₄-C₁₂ alkyl)acrylamides, such as N-octylacrylamide;

and mixtures thereof.

Among these monomers with a T_{g} less than 20° C., non-limiting mention may for example be made of methyl acrylate, ethyl acrylate, isobutyl acrylate, 2-ethylhexyl (meth)acrylate and mixtures thereof.

In at least one embodiment, all the monomers other than the silicone monomers are chosen from (meth)acrylic acid esters and (meth)acrylic acid.

The weight-average molecular weight (M_{w}) of the polymer according to the present disclosure is, in at least one embodiment, in a range from 25,000 to 1,000,000, for example from 30,000 to 750,000, such as from 40,000 to 500,000 and for example from 50,000 to 250,000.

The weight-average molecular weights (M_{w}) and number-average molecular weights (Mₙ) may be determined by gel permeation chromatography (solvent: THF, calibration curve established with linear polystyrene standards, refractive index and UV detector).

In at least one embodiment, the polydispersity index of the polymer according to the present disclosure is greater than 2, for example ranging from 2 to 9, such as greater than or equal to 2.5, for example ranging from 2.5 to 8 and such as ranging from 2.8 to 7. The polydispersity index Iₚ of the polymer is equal to the ratio of the weight-average molecular weight M_{w} to the number-average molecular weight Mₙ.

The polymer according to the present disclosure may be obtained by radical polymerization in solution according to the following preparation process:

some of the polymerization solvent can be introduced into a suitable reactor, heated until reaching the temperature suitable for the polymerization (typically from 60 to 120° C.);

once this temperature is reached, the constituent monomers of the first block can be added, in the presence of some of the polymerization initiator;

at the end of a time T corresponding to a maximum degree of conversion of for example 90%, the constituent monomers of the second block and the other part of the initiator can be introduced; and

the mixture is left to react for a time T′ (ranging, for example, from 3 to 6 hours), at the end of which the mixture is brought to ambient temperature (25° C.), so as to obtain the polymer in solution in the polymerization solvent.

As used herein, the expression “polymerization solvent” is understood to mean a solvent, or a mixture of solvents, for example chosen from ethyl acetate, butyl acetate, C₁-C₆ alcohols such as isopropanol and ethanol, aliphatic alkanes such as isododecane and mixtures thereof. In at least one embodiment, the polymerization solvent is a mixture of butyl acetate and isopropanol or is isododecane.

In at least one embodiment, the polymer according to the present disclosure is not water-soluble, i.e. the polymer is not soluble in water or in a mixture of water and linear or branched monoalcohols having from 2 to 5 carbon atoms such as ethanol, isopropanol or n-propanol, without pH modification, at a content of active material of at least 1 wt %, at ambient temperature (25° C.).

The present disclosure also relates to compositions, for example cosmetic or pharmaceutical compositions, comprising at least one polymer as described above, in a physiologically, such as cosmetically or pharmaceutically, acceptable medium.

The polymers according to the present disclosure may be present, alone or as a mixture, in the compositions according to the present disclosure in an amount ranging from 0.01 to 50% by weight, for example from 0.05 to 40% by weight, such as from 0.1 to 30% by weight, for example from 0.5 to 20% by weight, such as from 1 to 15% by weight, for example from 1.5 to 12% by weight, relative to the total weight of the composition.

They may be present in the composition in dissolved form or in the form of a dispersion, for example in an oil or a carbon-based organic solvent, such as isododecane.

For example, the polymers according to the present disclosure may be soluble or dispersible in at least one of the phases of the composition which comprises them, at a content of at least 1% by weight, at ambient pressure and temperature (25° C., 1 atm). They may be, for example, soluble in isododecane at a content of at least 1% by weight, such as at least 10% by weight, at ambient pressure and temperature.

The cosmetic or pharmaceutical compositions according to the present disclosure comprise, besides said polymers, a physiologically acceptable, for example cosmetically or pharmaceutically acceptable, medium, e.g. a medium that is compatible with keratinous substances such as the skin of the face or of the body, the hair, eyelashes, eyebrows and nails.

The composition may thus comprise a hydrophilic medium comprising water or a mixture of water and at least one hydrophilic organic solvent such as alcohols and for example linear or branched lower monoalcohols having from 2 to 5 carbon atoms such as ethanol, isopropanol or n-propanol, and polyols such as glycerine, diglycerine, propylene glycol, sorbitol, pentylene glycol and polyethylene glycols, hydrophilic C₂-C₄ aldehydes and C₂ ethers.

The water or mixture of water and at least one hydrophilic organic solvent may be present in the composition according to the present disclosure in an amount ranging from 0.1% to 99% by weight, relative to the total weight of the composition, for example from 10% to 80% by weight.

The composition may also be anhydrous.

The composition may also comprise a fatty phase which may comprise fatty substances that are liquid at ambient temperature (generally 25° C.) and/or fatty substances that are solid at ambient temperature such as waxes, pasty fatty substances, gums and mixtures thereof. These fatty substances may be of animal, vegetable, mineral or synthetic origin. This fatty phase may, in addition, comprise lipophilic organic solvents.

Regarding the fatty substances that are liquid at ambient temperature, often called oils, that can be used in the present disclosure, non-limiting mention may be made of: hydrocarbon-based oils of animal origin such as perhydrosqualene; hydrocarbon-based vegetable oils such as liquid triglycerides of fatty acids having from 4 to 10 carbon atoms such as the triglycerides of heptanoic or octanoic acids, or sunflower, maize, soybean, grape seed, sesame, apricot, macadamia, castor or avocado oils, triglycerides of caprylic/capric acids, jojoba or shea butter oil; linear or branched hydrocarbons of mineral or synthetic origin such as paraffin oils and derivatives thereof, vaseline, polydecenes, hydrogenated polyisobutene such as parleam; synthetic esters and ethers for example of fatty acids such as, for example, purcellin oil, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, and heptanoates, octanoates or decanoates of fatty alcohols; polyol esters such as propylene glycol dioctanoate, neopentyl glycol diheptanoate, diethylene glycol diisononanoate; and pentaerythritol esters; fatty alcohols having from 12 to 26 carbon atoms such as octyldodecanol, 2-butyloctanol, 2-hexyldecanol, 2-undecylpentadecanol, and oleyl alcohol; partially hydrocarbon-based and/or silicone-based fluoro oils; silicone oils such as linear or cyclic, volatile or non-volatile polymethylsiloxanes (PDMSs) that are liquid or pasty at ambient temperature such as cyclomethicones, dimethicones, optionally comprising a phenyl group, such as phenyltrimethicones, phenyltrimethylsiloxydiphenylsiloxanes, diphenyl-methyldimethyltrisiloxanes, diphenyldimethicones, phenyldimethicones, polymethylphenylsiloxanes; and mixtures thereof. These oils may be present in an amount ranging from 0.01 to 90%, for example from 0.1 to 85% by weight, relative to the total weight of the composition.

The composition according to the present disclosure may also comprise at least one physiologically acceptable organic solvent.

These solvents may be present in an amount ranging from 0.1 to 90%, such as from 0.5 to 85%, and for example from 10 to 80% by weight, relative to the total weight of the composition, such as from 30 to 50%.

Mention may for example be made, besides the hydrophilic organic solvents mentioned above, of ketones that are liquid at ambient temperature such as methyl ethyl ketone, methyl isobutyl ketone, diisobutyl ketone, isophorone, cyclohexanone, or acetone; propylene glycol ethers that are liquid at ambient temperature such as propylene glycol monomethyl ether, propylene glycol monomethyl ether acetate, dipropylene glycol mono-n-butyl ether; short-chain esters (having from 3 to 8 carbon atoms in total) such as ethyl acetate, methyl acetate, propyl acetate, n-butyl acetate, and isopentyl acetate; ethers that are liquid at 25° C. such as diethyl ether, dimethyl ether or dichlorodiethyl ether; alkanes that are liquid at 25° C. such as decane, heptane, dodecane, isododecane and cyclohexane; cyclic aromatic compounds that are liquid at 25° C. such as toluene and xylene; aldehydes that are liquid at 25° C. such as benzaldehyde, acetaldehyde and mixtures thereof.

The term “wax,” as used herein, is understood to mean a lipophilic compound that is solid at ambient temperature (25° C.) has a reversible solid/liquid phase change, and which has: a melting point ranging from 25° C. to 120° C. On bringing the wax to the liquid state (melting), it is possible to make it miscible with the oils that are optionally present and to form a microscopically homogeneous mixture but, on bringing the temperature of the mixture back to ambient temperature, recrystallization of the wax in the oils of the mixture is obtained. The melting point of the wax may be measured using a differential scanning calorimeter (DSC), for example the calorimeter sold under the name DSC 30 by Mettler.

The waxes may be hydrocarbon-based, fluoro and/or silicone waxes and may be of vegetable, mineral, animal and/or synthetic origin. For example, the waxes may have a melting point greater than 30° C., such as a melting point greater than 45° C. As a wax that can be used in the composition of the present disclosure, non-limiting mention may be made of beeswax, carnauba wax, candelilla wax, paraffin wax, microcrystalline waxes, ceresin or ozokerite; synthetic waxes such as polyethylene or Fischer-Tropsch waxes, silicone waxes such as alkyl or alkoxy dimethicones having from 16 to 45 carbon atoms.

The gums may be high-molecular weight polydimethylsiloxanes (PDMSs) or cellulose gums or polysaccharides and the pasty substances may be hydrocarbon-based compounds such as lanolins and derivatives thereof or PDMSs.

As used herein, the expression “pasty fatty substance” is understood to mean a viscous product comprising a liquid fraction and a solid fraction. For example, it is understood to mean fatty substances having a melting point ranging from 20 to 55° C., for example from 25 to 45° C., and/or a viscosity at 40° C. ranging from 0.1 to 40 Pa·s (from 1 to 400 poise), such as from 0.5 to 25 Pa·s, measured with a Contraves TV or Rhoomat 80. A person skilled in the art may choose the spindle that makes it possible to measure the viscosity from the spindles MS-r3 and MS-r4, based on his or her general knowledge, so as to be able to carry out the measurement of the viscosity of the pasty compound tested.

The melting point values correspond, according to the present disclosure, to the melting peak measured by the differential scanning calorimetry method with a temperature rise of 5 or 10° C./min.

In at least one embodiment, these fatty substances are hydrocarbon-based compounds (mainly comprising carbon and hydrogen atoms and optionally ester groups), optionally of polymeric type; they may also be chosen from silicone and/or fluoro compounds; they may also be in the form of a mixture of hydrocarbon-based and/or silicone and/or fluoro compounds. In the case of a mixture of various pasty fatty substances, use may predominantly be made, for example, of pasty hydrocarbon-based compounds.

Among the pasty compounds likely to be used in the composition according to the present disclosure, non-limiting mention may be made of lanolins and lanolin derivatives such as acetylated lanolins or oxypropylenated lanolins or isopropyl lanolate; esters of fatty acids or alcohols, for example those having from 20 to 65 carbon atoms such as triisostearyl or cetyl citrate; arachidyl propionate; poly(vinyl laurate); cholesterol esters such as triglycerides of vegetable origin such as hydrogenated vegetable oils, viscous polyesters such as poly(12-hydroxystearic acid) and mixtures thereof. As triglycerides of vegetable origin, it is possible to use the derivatives of hydrogenated castor oil.

Non-limiting mention may also be made of silicone pasty fatty substances such as polydimethylsiloxanes (PDMSs) having alkyl or alkoxy type pendant chains that have from 8 to 24 carbon atoms, such as stearyl dimethicones.

The nature and amount of solid substances are a function of the desired mechanical properties and textures. By way of indication, the composition may comprise from 0.1 to 50% by weight of waxes, relative to the total weight of the composition and for example from 1 to 30% by weight.

The composition according to the present disclosure may moreover comprise, in one phase, pigments and/or pearlescent agents and/or fillers commonly used in cosmetic compositions.

The composition may also comprise other coloring substances chosen from water-soluble dyes and/or liposoluble dyes well known to a person skilled in the art.

The term “pigments,” as used herein, should be understood to mean white or colored, mineral or organic particles of any shape which are insoluble in the physiological medium, and which are intended to color the composition.

The term “fillers,” as used herein, should be understood to mean colorless or white, mineral or synthetic lamellar or non-lamellar particles intended to give body or stiffness to the composition and/or softness, mattness and uniformity to the makeup.

The phrase “pearlescent agents,” as used herein, should be understood to mean iridescent particles of any shape, for example those produced by certain molluscs in their shell or else synthesized.

The pigments may be present in the composition in an amount ranging from 0.01 to 25% by weight of the final composition, and for example in an amount ranging from 3 to 10% by weight. They may be white or colored, mineral or organic. Non-limiting mention may be made of titanium, zirconium or cerium oxides, and also zinc, iron or chromium oxides, ferric blue, chromium hydrate, carbon black, ultramarines (aluminosilicate polysulphides), manganese pyrophosphate and certain metal powders such as those of silver or aluminium. Non-limiting mention may also be made of D&C pigments and lacquers, commonly used to give a makeup effect to the lips and skin, which are calcium, barium, aluminium, strontium or zirconium salts.

The pearlescent agents may be present in the composition in an amount ranging from 0.01 to 20% by weight, for example ranging from 3 to 10% by weight. Among the pearlescent agents which can be envisaged, non-limiting mention may be made of natural mother-of-pearl, mica covered with titanium oxide, with iron oxide, with natural pigment or with bismuth oxychloride, and also colored titanium oxide-coated mica.

Among the liposoluble or water-soluble dyes, which may be present in the composition, alone or as a mixture, in an amount ranging from 0.001 to 15% by weight, such as from 0.01 to 5% by weight, and for example from 0.1 to 2% by weight, relative to the total weight of the composition, non-limiting mention may be made of the disodium salt of ponceau, the disodium salt of alizarine green, quinoline yellow, the trisodium salt of amaranth, the disodium salt of tartrazine, the monosodium salt of rhodamine, the disodium salt of fuchsin, zanthophyll, methylene blue, cochineal carmine, haloacid, azo or anthraquinone dyes, copper or iron sulphate, Sudan brown, Sudan red and annatto, and also beet juice and carotene.

The composition according to the present disclosure may, in addition, comprise at least one filler, for example in an amount ranging from 0.01% to 50% by weight, relative to the total weight of the composition, such as ranging from 0.02% to 30% by weight. The fillers may be mineral or organic and of any shape, platelet, spherical or oblong. Non-limiting mention may be made of talc, mica, silica, kaolin, polyamide (Nylon®) powders, poly(β-alanine) powders, polyethylene powders, tetrafluoroethylene polymer (Teflon®) powders, lauroyl lysine, starch, boron nitride, polymeric hollow microspheres such as those made of polyvinylidene chloride/acrylonitrile such as Expancel® (Nobel Industrie), made of acrylic acid copolymers (Polytrap® from Dow Corning) and silicone resin microbeads (Tospearls® from Toshiba, for example), polyorganosiloxane elastomer particles, precipitated calcium carbonate, magnesium carbonate and hydrogen carbonate, hydroxyapatite, hollow silica microspheres (Silica Beads® from Maprecos), glass or ceramic microcapsules, metal soaps derived from organic carboxylic acids having from 8 to 22 carbon atoms, such as from 12 to 18 carbon atoms, for example zinc, magnesium or lithium stearate, zinc laurate or magnesium myristate.

The composition may moreover comprise an additional polymer such as a film-forming polymer. According to the present disclosure, the expression “film-forming polymer” is understood to mean a polymer capable of forming, by itself alone or in the presence of an additional film-forming agent, a continuous and adherent film on a support, especially on keratinous substances. Among the film-forming polymers likely to be used in the composition of the present disclosure, non-limiting mention may be made of synthetic polymers of radical type or of polycondensate type, polymers of natural origin and mixtures thereof, for example acrylic polymers, polyurethanes, polyesters, polyamides, polyureas, and cellulose polymers such as nitrocellulose.

The composition according to the present disclosure may also comprise ingredients commonly used in cosmetics, such as vitamins, thickeners, gelling agents, trace elements, demulcents, sequestering agents, fragrances, basifying agents, acidifying agents, preservatives, sunscreens, surfactants, antioxidants, agents for combating hair loss, antidandruff agents, propellants, ceramides, auxiliary film-forming agents, or mixtures thereof.

Of course, a person skilled in the art will be sure to choose any optional additional compounds, and/or their amounts, so that the advantageous properties of the composition according to the present disclosure are not, or are not substantially, impaired by the envisaged addition.

The composition according to the present disclosure may be present in the form of a suspension, a dispersion, for example of oil in water, due to vesicles; an optionally thickened, or even gelled, aqueous or oily solution; an oil-in-water, water-in-oil, or multiple emulsion; a gel or a foam; an oily or emulsified gel; a dispersion of vesicles, for example lipid vesicles; a two-phase or multiphase lotion; a spray; a loose, compact or cast powder; or an anhydrous paste. This composition may have the appearance of a lotion, a cream, an ointment, a soft paste, a salve, a foam, or a cast or moulded solid, for example in stick or dish form, or else a compacted solid.

A person skilled in the art will be able to choose the appropriate galenic form, and also its preparation method, based on his general knowledge, taking into account, on the one hand, the nature of the constituents used, especially their solubility in the support and, on the other hand, the application envisaged for the composition.

The cosmetic composition according to the present disclosure may be in the form of a care and/or makeup product for the skin of the body or face, the lips, nails, eyelashes, eyebrows and/or hair, of a suntan or self-tanning product, of a hair product for the care, treatment, shaping, makeup or dyeing of the hair.

It may thus be in the form of a makeup composition, for example a product for the complexion such as a foundation, face powder or eye shadow; a product for the lips such as a lipstick or a lip salve; a concealer, a blusher, a mascara, an eyeliner, a makeup product for the eyebrows, a lip or eye pencil; a product for the nails such as a nail varnish or nail care product; a body makeup product; a hair makeup product (mascara or lacquer for hair).

It may also be in the form of a composition for protecting or caring for the skin of the face, neck, hands or body, for example an anti-wrinkle composition, a moisturizing or treating composition; an anti-sun composition or artificial tanning composition.

It may also be in the form of a hair product, for example for dyeing, form retention of the hairstyle, hair shaping, care, treatment or cleaning of hair, such as shampoos, gels, hair setting lotions, lotions for blow-drying, or fixing and styling compositions such as lacquers or spray.

In at least one embodiment, the cosmetic composition according to the present disclosure is present in the form of a makeup product, for example a lipstick.

The present disclosure also relates to cosmetic treatment methods, for example for making up or caring for the keratinous substances such as the skin of the body or face, the lips, nails, eyelashes, eyebrows and/or hair, comprising the application to said substances of a cosmetic composition as defined previously.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

The examples which follow serve to illustrate the invention without being limiting.

### EXAMPLES

### Gloss Measured with a Gloss Meter on a Dry Deposited Layer of Polymer

The gloss can be measured conventionally by the following method using a gloss meter.

A 50 μm thick layer of a 50% solution of the polymer in isododecane is spread, using an automatic spreader, over a Leneta contrast chart with the reference Form 1A Penopac. The layer covers at least the black background of the chart. The deposited layer is left to dry for 24 hours at a temperature of 25° C. and then the 20° gloss is measured on the black background using a Dr Lange Ref03 gloss meter.

A measurement at 20° that is greater than around 50 gives a gloss that is judged to be acceptable and that is very satisfactory when the measurement is greater than 60.

### Oil Resistance of the Polymers by Measurement of the Tackiness

Oil resistance is determined with a drop of olive oil placed on a dry polymer film.

A polymer film is produced from a 20% solution of polymer in isododecane; 0.5 ml is spread over a 2.5×7.5 cm glass plate and left to dry at ambient temperature (25° C.) for 24 hours. Next, 1 ml of olive oil is spread over the polymer film.

After 1 hour, the excess oil is wiped from the film and the tackiness is evaluated by touch, in comparison with the comparative polymer.

A grade of “***” or “3*” is given when the sheet is unstuck with a finger (very tacky).

A grade of “0” means: no tack detected.

The tack reflects the sensitivity of the polymer to the olive oil. The tackier the polymer is in the presence of oil, the more it is sensitive to the oil and therefore the more the deposit will be easily impaired, for example during meals (in the presence of food oil) or by sebum. This results in a worse hold of the polymer on the skin. It also results in a decrease in comfort: the tackier the film is, the more the composition will be uncomfortable to wear.

### IS: Internal Stresses

A polymer film is produced from a 20% solution of polymer in isododecane; 30 ml are spread over a 6×1 cm nitrile strip and left to dry at ambient temperature (25° C.) for 3 hours. The deformation of the support is observed visually after drying.

A grade of 0 means that the support is not deformed during drying.

A grade of +++ means that the support is almost bent (completely deformed) after drying.

The internal stresses (IS) reflect the comfort from the mechanical point of view: when there is no deformation of the support during drying, it can be expected that there will be no discomfort during drying of the composition after its application to the skin.

### Example 1

Introduced into a 1 liter reactor were 300 g of isododecane, then the temperature was increased so as to pass from ambient temperature (25° C.) to 90° C. over 1 hour. Added next, at 90° C. and over 1 hour, were 105 g of isobornyl acrylate, 105 g of isobornyl methacrylate and 1.8 g of 2,5-bis(2-ethylhexanoylperoxy)-2,5-dimethylhexane (Trigonox® 141 from Akzo Nobel). The mixture was kept at 90° C. for 1 h 30 min.

Introduced next to the preceding mixture, still at 90° C. and over 30 minutes were 82.5 g of isobutyl acrylate, 7.5 g of methacryloxypropyltrimethoxysilane (MPS) and 1.2 g of 2,5-bis(2-ethylhexanoylperoxy)-2,5-dimethylhexane. The mixture was kept at 90° C. for 3 hours, then the whole mixture was cooled.

A solution comprising 50.8% of polymer active material in isododecane was obtained. The polymer comprised a first block comprising the isobornyl (meth)acrylates and a second block comprising the silicone monomer and the isobutyl acrylate.

### Example 2

Introduced into a 1 liter reactor were 300 g of isododecane, then the temperature was increased so as to pass from ambient temperature (25° C.) to 90° C. over 1 hour. Added next, at 90° C. and over 1 hour, were 105 g of isobornyl acrylate, 105 g of isobornyl methacrylate and 1.8 g of 2,5-bis(2-ethylhexanoylperoxy)-2,5-dimethylhexane (Trigonox® 141 from Akzo Nobel). The mixture was kept at 90° C. for 1 h 30 min.

Introduced next to the preceding mixture, still at 90° C. and over 30 minutes were 82.5 g of isobutyl acrylate, 7.5 g of methacryloxypropyltris-(trimethylsiloxy)silane (MPTS) and 1.2 g of 2,5-bis(2-ethylhexanoylperoxy)-2,5-dimethylhexane. The mixture was kept at 90° C. for 3 hours, then the whole mixture was cooled.

A solution comprising 50.8% of polymer active material in isododecane was obtained. The polymer comprised a first block comprising the isobornyl (meth)acrylates and a second block comprising the silicone monomer and the isobutyl acrylate.

### Example 3

Introduced into a 1 liter reactor were 300 g of isododecane, then the temperature was increased so as to pass from ambient temperature (25° C.) to 90° C. over 1 hour. Added next, at 90° C. and over 1 hour, were 97.5 g of isobornyl acrylate, 105 g of isobornyl methacrylate, 7.7 g of methacryloxypropyltris(trimethylsiloxy)silane (MPTS) and 1.8 g of 2,5-bis(2-ethylhexanoylperoxy)-2,5-dimethylhexane (Trigonox® 141 from Akzo Nobel). The mixture was kept at 90° C. for 1 h 30 min.

Introduced next to the preceding mixture, still at 90° C. and over 30 minutes were 90 g of isobutyl acrylate and 1.2 g of 2,5-bis(2-ethylhexanoylperoxy)-2,5-di-methylhexane. The mixture was kept at 90° C. for 3 hours, then the whole mixture was cooled.

A solution comprising 49.2% of polymer active material in isododecane was obtained. The polymer comprised a first block comprising the isobornyl (meth)acrylates and the silicone monomer and a second block comprising the isobutyl acrylate.

### Comparative Example

### Outside of the Invention

Introduced into a 1 liter reactor were 100 g of isododecane, then the temperature was increased so as to pass from ambient temperature (25° C.) to 90° C. over 1 hour. Added next, at 90° C. and over 1 hour, were 105 g of isobornyl acrylate, 105 g of isobornyl methacrylate, 110 g of isododecane and 1.8 g of 2,5-bis(2-ethylhexanoyl-peroxy)-2,5-dimethylhexane (Trigonox® 141 from Akzo Nobel). The mixture was kept at 90° C. for 1 h 30 min.

Introduced next to the preceding mixture, still at 90° C. and over 30 minutes were 90 g of isobutyl acrylate, 90 g of isododecane and 1.2 g of 2,5-bis(2-ethyl-hexanoylperoxy)-2,5-dimethylhexane. The mixture was kept at 90° C. for 3 hours, then the whole mixture was cooled.

A solution comprising 50% of polymer active material in isododecane was obtained. The polymer comprised a first block comprising the isobornyl (meth)acrylates and a second block comprising the isobutyl acrylate.

### Example 4

The films obtained from the polymers of Examples 1 to 3 and also with the comparative polymer prepared above, were characterized:

Film: the brittle nature of a single film of polymer was observed visually.

Also determined, according to the methods indicated above, were the tack (resistance to olive oil) and the internal stresses (IS) of the polymer.

The following results were obtained:

| | Example 1 | Example 2 | Example 3 | Comparative |
|---|---|---|---|---|
| % Isob. A. | 35 | 35 | 32.5 | 35 |
| % Isob. M. | 35 | 35 | 35 | 35 |
| % MPTS | — | 2.5 | 2.5 | — |
| % MPS | 2.5 | — | — | — |
| % Ibut. A. | 27.5 | 27.5 | 30 | 30 |
| M_{w} (g/mol) | 92,800 | 109,800 | 128,900 | 100,300 |
| Ip | 3.97 | 3.26 | 3.98 | 4.40 |
| SC(%) | 50.8 | 50.8 | 49.2 | 50.0 |
| Single film | slightly | slightly | barely | brittle |
| (visual | brittle | brittle | brittle | |
| observation) | | | | |
| Gloss at 20° | 76.2 | 66.1 | 60.8 | 71.3 |
| Tack | 0 | 0 | +++ | +++ |
| Internal | ++ | ++ | ++ | +++ |
| stresses (IS) | | | | |

Isob. A.: isobornyl acrylate
Isob. M.: isobornyl methacrylate
MPTS: methacryloxypropyltris(trimethylsiloxy)silane
MPS: methacryloxypropyltrimethoxysilane
Ibut. A.: isobutyl acrylate
SC: solids content

### Example 5

A lipstick was prepared comprising (% by weight):

| | |
|---|---|
| polyethylene wax | 15% |
| solution of the polymer from Example 1 | 20% |
| comprising 50.8% of dry matter in isododecane | |
| hydrogenated polyisobutene (parleam from | 25% |
| Nippon Oil Fats) | |
| pigments | 10% |
| isododecane | qs for 100% |

The composition obtained after application to the lips had good cosmetic properties.

1-22. (canceled)

23. A block copolymer comprising at least one first block and at least one second block, wherein:
the at least one first block comprises from 0.5 to 35% by weight, relative to the weight of said first block, of at least one silicone monomer chosen from, alone or as a mixture, the following monomers:
(i) ethylenic monomers of which the ester group comprises silanes and/or siloxanes, of formula: 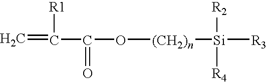
wherein:
R₁ is chosen from a hydrogen atom and a methyl group;
R₂, R₃, and R₄, which are identical or different, are chosen from C₁-C₆ alkyl groups and —OSi(R₅)₃ groups wherein R₅ is chosen from methyl and ethyl groups;
n is an integer from 1 to 10;
(ii) polydimethylsiloxanes macromonomers, or polydimethylsiloxanes having a monoacryloyloxy or monomethacryloyloxy terminal group, of the following formula: 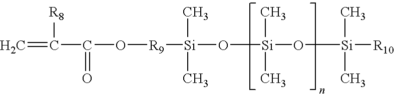
wherein:
R₈ is chosen from a hydrogen atom and a methyl group;
R₉ is chosen from linear and branched divalent hydrocarbon-based groups having from 1 to 10 carbon atoms and optionally comprising one or two ether —O— bonds;
R₁₀ is chosen from linear and branched alkyl groups having from 1 to 10 carbon atoms;
n is an integer ranging from 1 to 300;
(iii) ethylenic monomers of which the ester group comprises carboxysilane dendrimers of formula: 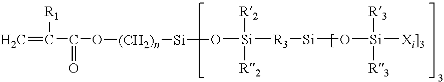
wherein:
R₁ is chosen from a hydrogen atom and a methyl group;
n is an integer from 1 to 10;
R′₂, R″₂, R′₃ and R″₃, which are identical or different, are chosen from C₁ to C₁₀ alkyl groups;
R₃ is chosen from C₂ to C₁₀ alkylene divalent groups;
i is an integer from 1 to 10;
provided that when i is an integer from 2 to 10, X₍ᵢ₎ which are identical or different, is chosen from —R₄—Si—[O—(R′₃)(R″₃)—X₍ᵢ₋₁₎]₃ groups wherein R₄, being identical or different, is chosen from C₂ to C₁₀ alkylene divalent groups; and
when i is 1, X₍ᵢ₎ is chosen from a hydrogen atom and C₁-C₁₀ alkyl groups;
(iv) ethylenic monomers of polyhedral oligomeric silsesquioxanes (POSS) or polyhedral oligomeric silicates (POS) type of structure: 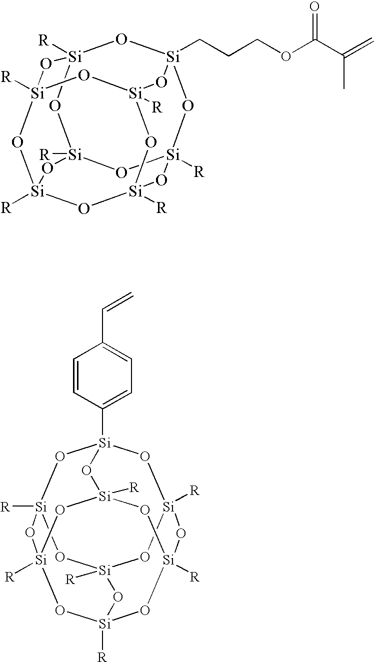
wherein R, being identical or different, is chosen from linear C₁ to C₁₀ alkyl groups and cyclic C₃ to C₁₂ alkyl groups; and
the at least one second block comprises from 0.5 to 100% by weight, relative to the weight of said second block, of at least one monomer chosen from, alone or as a mixture, monomers of formula (I): CH₂═C(R₁)—COOR₂, wherein R₁ is chosen from a hydrogen atom and a methyl group, and R₂ is chosen from C₄ to C₁₂ cycloalkyl groups,
wherein the at least one monomer of formula (I) is present in an amount ranging from 0.5 to 99.5% by weight, of the total weight of the block comprising it, and
wherein the at least one silicone monomer and the at least one monomer chosen from monomers of formula (I) are in the same block.

24. The block copolymer according to claim 23, wherein the at least one silicone monomer is chosen from, alone or as a mixture:
(meth)acryloxypropyltris(trimethylsiloxy)silane,
(meth)acryloxypropylbis(trimethylsiloxy)methylsilane,
(meth)acryloxymethyltris(trimethylsiloxy)silane,
(meth)acryloxymethylbis(trimethylsiloxy)methylsilane;
(meth)acryloxypropyltrimethoxysilane; and
the following monomers, wherein R₁ is chosen from a hydrogen atom and a methyl group:

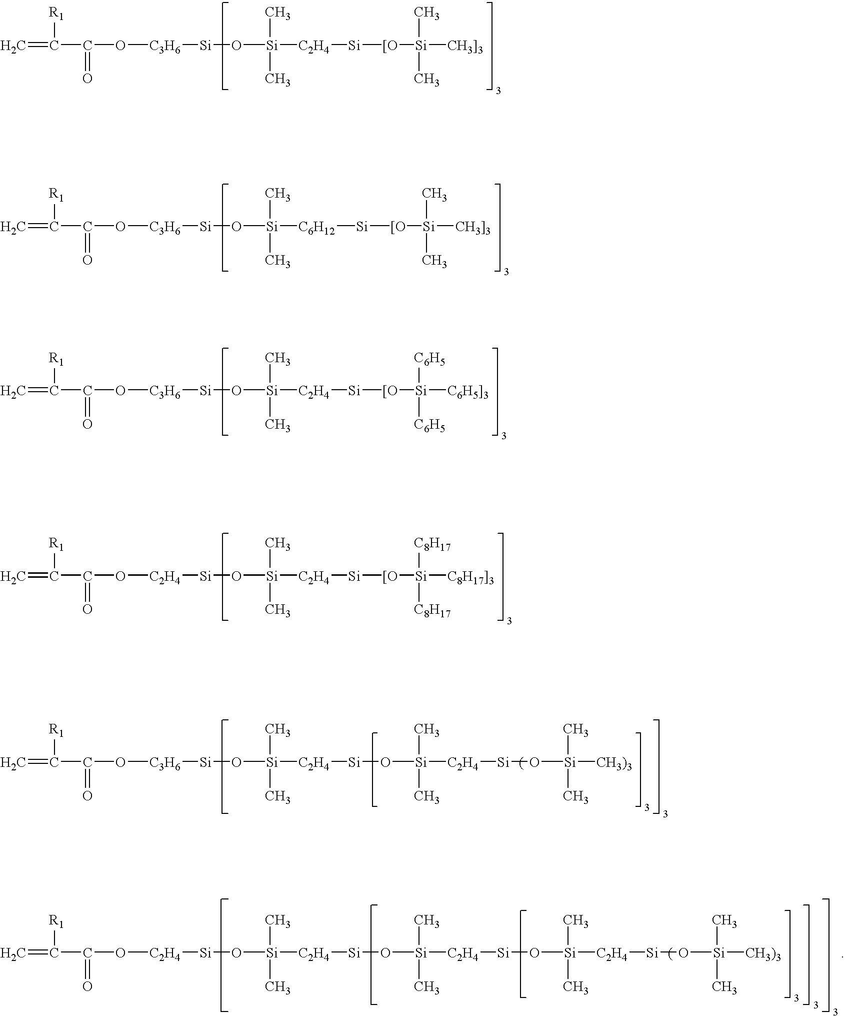

25. The block copolymer according to claim 23, wherein the at least one silicone monomer is present in an amount ranging from 0.5% to 35% by weight, of the total weight of the block comprising said at least one silicone monomer.

26. The block copolymer according to claim 23, wherein the monomers of formula (I) are chosen from isobornyl methacrylate, isobornyl acrylate, cyclohexyl acrylate, cyclohexyl methacrylate, t-butylcyclohexyl acrylate, t-butylcyclohexyl methacrylate, and mixtures thereof.

27. The block copolymer according to claim 23, further comprising at least one additional monomer, chosen from, alone or as a mixture:
(i) ethylenic hydrocarbons having from 2 to 10 carbons;
(ii) (meth)acrylates of formula CH₂═CHCOOR′₃ or CH₂═C(CH₃)COOR′₃ wherein R′₃ is chosen from:
linear and branched alkyl groups having from 1 to 22 carbon atoms, optionally interrupted by at least one heteroatom chosen from O, N, S and P; said alkyl group being optionally substituted by at least one substituent chosen from hydroxyl groups and halogen atoms;
C₃ to C₂₀ aryl groups;
C₄ to C₃₀ aralkyl groups (C₁ to C₈ alkyl groups);
heterocyclic groups having from 4 to 12 chain members comprising at least one heteroatom chosen from O, N and S, wherein the ring is aromatic or non-aromatic;
heterocycloalkyl groups (C₁-C₄ alkyls), said aryl, aralkyl, heterocyclic and heterocycloalkyl groups being optionally substituted by at least one substituent chosen from hydroxyl groups, halogen atoms, and linear and branched C₁₋₄ alkyl groups, optionally interrupted by at least one heteroatom chosen from O, N, S and P, said alkyl groups being optionally substituted by at least one substituent chosen from hydroxyl groups and halogen atoms,
groups of the formula —(C₂H₄O)ₘ—R″, wherein m is an integer from 5 to 150 and R″ is chosen from a hydrogen atom and C₁ to C₃₀ alkyl groups;
(iii) (meth)acrylamides of formula: 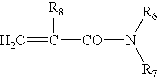
wherein R₈ is chosen from a hydrogen atom and a methyl group; and R₇ and R₆, which are identical or different, are chosen from:
hydrogen atoms; or
linear and branched alkyl groups having from 1 to 22 carbon atoms, optionally interrupted by at least one heteroatom chosen from O, N, S and P; said alkyl group being optionally substituted by at least one substituent chosen from hydroxyl groups, and halogen atoms;
C₃ to C₁₂ cycloalkyl groups;
C₃ to C₂₀ aryl groups;
C₄ to C₃₀ aralkyl groups (C₁ to C₅ alkyl groups);
heterocyclic groups having from 4 to 12 chain members comprising at least one heteroatom chosen from O, N and S, wherein the ring is aromatic or non-aromatic;
heterocycloalkyl groups, said alkyl groups comprising 1 to 4 carbon atoms, said cycloalkyl, aryl, aralkyl, heterocyclic and heterocycloalkyl groups being optionally substituted by at least one substituent chosen from hydroxyl groups, halogen atoms, and linear and branched C₁-C₄ alkyl groups, optionally interrupted by at least one heteroatom chosen from O, N, S and P, said alkyl groups being optionally substituted by at least one substituent chosen from hydroxyl groups and halogen atoms;
(iv) vinyl compounds of formulae:
CH₂═CH—R₉, CH₂═CH—CH₂—R₉ and CH₂═C(CH₃)—CH₂—R₉
wherein R₉ is chosen from a hydroxyl group, Cl and F atoms, NH₂, OR₁₄ wherein R₁₄ is chosen from a phenyl group and C₁ to C₁₂ alkyl groups; acetamide (NHCOCH₃); an OCOR₁₅ group wherein R₁₅ is chosen from linear and branched alkyl groups having from 2 to 12 carbons; and a group chosen from:
linear and branched alkyl groups having from 1 to 22 carbon atoms, optionally interrupted by at least one heteroatom chosen from O, N, S and P; said alkyl group being optionally substituted by at least one substituent chosen from hydroxyl groups, and halogen atoms;
C₃ to C₁₂ cycloalkyl groups;
C₃ to C₂₀ aryl groups;
C₄ to C₃₀ aralkyl groups (C₁ to C₅ alkyl groups);
heterocyclic groups having 4 to 12 chain members comprising at least one heteroatom chosen from O, N and S, wherein the ring is aromatic or non-aromatic;
heterocycloalkyl groups (C₁-C₄ alkyls),
said cycloalkyl, aryl, aralkyl, heterocyclic and heterocycloalkyl groups being optionally substituted by at least one substituent chosen from hydroxyl groups, halogen atoms, and linear and branched C₁₋₄ alkyl groups, optionally interrupted by at least one heteroatom chosen from O, N, S and P, said alkyl groups being optionally substituted by at least one substituent chosen from hydroxyl groups and halogen atoms;
(v) monomers with at least one ethylenic unsaturation comprising at least one carboxylic, phosphoric or sulphonic acid or anhydride functional group and salts thereof;
(vi) monomers with at least one ethylenic unsaturation comprising at least one tertiary amine functional group and salts thereof.

28. The block copolymer according to claim 23, wherein the at least one block comprising the at least one monomer of formula (I) comprises both isobornyl methacrylate and isobornyl acrylate.

29. The block copolymer according to claim 23, wherein the at least one silicone monomer is present in a total amount ranging from 1 to 50% by weight, relative to the total weight of the polymer.

30. The block copolymer according to claim 23, wherein the at least one monomer of formula (I) is present in a total amount ranging from 20 to 80% by weight, relative to the total weight of the polymer.

31. The block copolymer according to claim 23, comprising a block having a T_{g} greater than or equal to 20° C.

32. The block copolymer according to claim 23, wherein the polymer exhibits a polydispersity index greater than 2.

33. A cosmetic composition comprising, in a physiologically acceptable medium, at least one block copolymer comprising at least one first block and at least one second block, wherein:
the at least one first block comprises from 0.5 to 35% by weight, relative to the weight of said first block, of at least one silicone monomer chosen from, alone or as a mixture, the following monomers:
(i) ethylenic monomers of which the ester group comprises silanes and/or siloxanes, of formula: 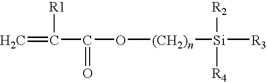
wherein:
R₁ is chosen from a hydrogen atom and a methyl group;
R₂, R₃, and R₄, which are identical or different, are chosen from C₁-C₆ alkyl groups and —OSi(R₅)₃ groups wherein R₅ is chosen from methyl and ethyl groups;
n is an integer from 1 to 10;
(ii) polydimethylsiloxanes macromonomers, or polydimethylsiloxanes having a monoacryloyloxy or monomethacryloyloxy terminal group, of the following formula: 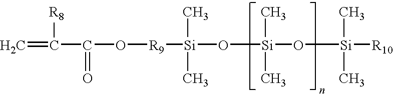
wherein:
R₈ is chosen from a hydrogen atom and a methyl group;
R₉ is chosen from linear and branched divalent hydrocarbon-based groups having from 1 to 10 carbon atoms and optionally comprising one or two ether —O— bonds;
R₁₀ is chosen from linear and branched alkyl groups having from 1 to 10 carbon atoms;
n is an integer ranging from 1 to 300;
(iii) ethylenic monomers of which the ester group comprises carboxysilane dendrimers of formula: 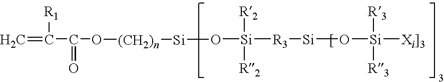
wherein:
R₁ is chosen from a hydrogen atom and a methyl group;
n is an integer from 1 to 10;
R′₂, R″₂, R′₃ and R″₃, which are identical or different, are chosen from C₁ to Cₐₒ alkyl groups;
R₃ is chosen from C₂ to C₁₀ alkylene divalent groups;
i is an integer from 1 to 10;
provided that
when i is an integer from 2 to 10, X₍ᵢ₎, which are identical or different, is chosen from —R₄—Si—[O—(R′₃)(R″₃)—X₍ᵢ₋₁₎]₃ groups wherein R₄, which are identical or different, is chosen from C₂ to C₁₀ alkylene divalent groups; and
when i is 1, X₍ᵢ₎ is chosen from a hydrogen atom and C₁-C₁₀ alkyl groups;
(iv) ethylenic monomers of polyhedral oligomeric silsesquioxanes (POSS) or polyhedral oligomeric silicates (POS) type of structure: 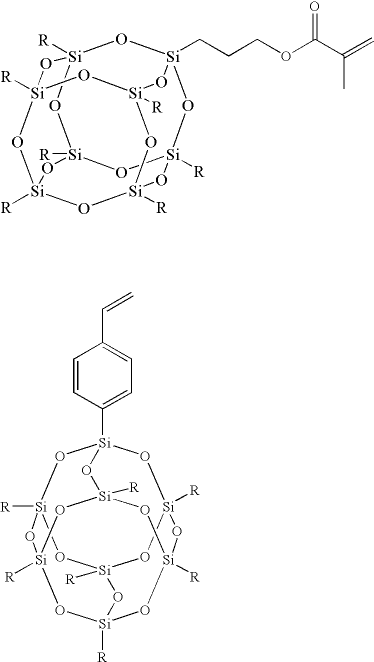
wherein R, being identical or different, is chosen from linear C₁ to C₁₀ alkyl groups and cyclic C₃ to C₁₂ alkyl groups; and
the at least second one block comprises from 0.5 to 100% by weight, relative to the weight of said second block, of at least one monomer chosen from, alone or as a mixture, monomers of formula (I): CH₂═C(R₁)—COOR₂, wherein R₁ is chosen from a hydrogen atom and a methyl group, and R₂ is chosen from C₄ to C₁₂ cycloalkyl groups,
wherein the at least one monomer of formula (I) is present in an amount ranging from 0.5 to 99.5% by weight, of the total weight of the block comprising it, and
wherein the at least one silicone monomer and the at least one monomer chosen from monomers of formula (I) are in the same block.

34. The composition according to claim 33, wherein the at least one block copolymer is present in an amount ranging from 0.01 to 50% by weight, relative to the total weight of the composition.

35. The composition according to claim 33, wherein the at least one block copolymer is present in dissolved form or in the form of a dispersion.

36. The composition according to claim 33, wherein the physiologically acceptable medium comprises at least one constituent chosen from water, hydrophilic organic solvents, waxes, pasty fatty substances, gums and mixtures thereof; lipophilic organic solvents; oils; pigments, pearlescent agents, fillers, water-soluble dyes, liposoluble dyes; polymers; vitamins, thickeners, gelling agents, trace elements, demulcents, sequestering agents, fragrances, basifying agents, acidifying agents, preservatives, sunscreens, surfactants, antioxidants, agents for combating hair loss, antidandruff agents, propellants, ceramides, auxiliary film-forming agents, and mixtures thereof.

37. The composition according to claim 33, wherein the composition is in a form chosen from a care and/or makeup product for the skin of the body or face, the lips, nails, eyelashes, eyebrows and/or hair; a suntan or self-tanning product; and a hair product for the care, treatment, shaping, makeup and/or dyeing of the hair.

38. The composition according to claim 33, wherein the composition is in the form of a makeup composition.

39. A cosmetic treatment method comprising applying to the skin of the body or face, the lips, nails, eyelashes, eyebrows and/or hair a cosmetic composition comprising, in a physiologically acceptable medium, at least one block copolymer comprising at least one first block and at least one second block, wherein:
the at least one first block comprises from 0.5 to 35% by weight, relative to the weight of said first block, of at least one silicone monomer chosen from, alone or as a mixture, the following monomers:
(i) ethylenic monomers of which the ester group comprises silanes and/or siloxanes, of formula: 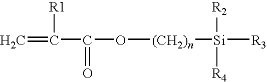
wherein:
R₁ is chosen from a hydrogen atom and a methyl group;
R₂, R₃, and R₄, which are identical or different, are chosen from C₁-C₆ alkyl groups and —OSi(R₅)₃ groups wherein R₅ is chosen from methyl and ethyl groups;
n is an integer from 1 to 10;
(ii) polydimethylsiloxanes macromonomers, or polydimethylsiloxanes having a monoacryloyloxy or monomethacryloyloxy terminal group, of the following formula: 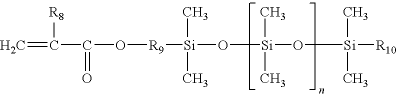
wherein:
R₈ is chosen from a hydrogen atom and a methyl group;
R₉ is chosen from linear and branched divalent hydrocarbon-based groups having from 1 to 10 carbon atoms and optionally comprising one or two ether —O— bonds;
R₁₀ is chosen from linear and branched alkyl groups having from 1 to 10 carbon atoms;
n is an integer ranging from 1 to 300;
(iii) ethylenic monomers of which the ester group comprises carboxysilane dendrimers of formula: 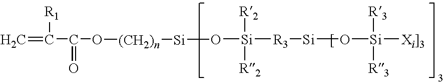
wherein:
R₁ is chosen from a hydrogen atom and a methyl group;
n is an integer from 1 to 10;
R′₂, R″₂, R′₃ and R″₃, which are identical or different, are chosen from C₁ to C₁₀ alkyl groups;
R₃ is chosen from C₂ to C₁₀ alkylene divalent groups;
i is an integer from 1 to 10;
provided that when i is an integer from 2 to 10, X₍ᵢ₎, which are identical or different, is chosen from —R₄—Si—[O—(R′₃)(R″₃)—X₍ᵢ₋₁₎]₃ groups wherein R₄, which are identical or different, is chosen from C₂ to C₁₀ alkylene divalent groups; and
when i is 1, X₍ᵢ₎ is chosen from a hydrogen atom and C₁-C₁₀ alkyl groups;
(iv) ethylenic monomers of polyhedral oligomeric silsesquioxanes (POSS) or polyhedral oligomeric silicates (POS) type of structure: 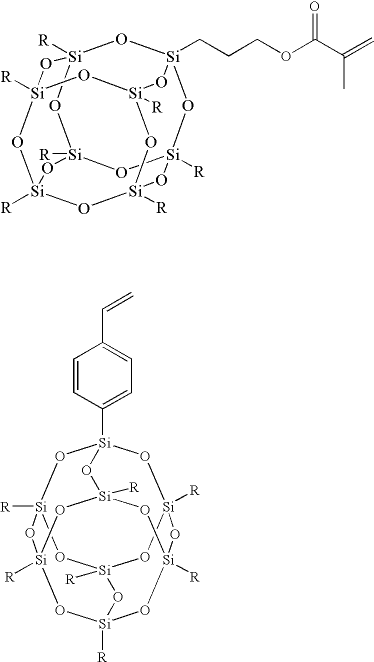
wherein R, which is identical or different, is chosen from linear C₁ to C₁₀ alkyl groups and cyclic C₃ to C₁₂ alkyl groups; and
the at least one second block comprises from 0.5 to 100% by weight, relative to the weight of said second block, of at least one monomer chosen from, alone or as a mixture, monomers of formula (I): CH₂═C(R₁)—COOR₂, wherein R₁ is chosen from a hydrogen atom and a methyl group, and R₂ is chosen from C₄ to C₁₂ cycloalkyl groups,
wherein the at least one monomer of formula (I) is present in an amount ranging from 0.5 to 99.5% by weight, of the total weight of the block comprising it, and
wherein the at least one silicone monomer and the at least one monomer chosen from monomers of formula (I) are in the same block.

40. The composition according to claim 39, wherein the at least one block copolymer is present in an amount ranging from 0.01 to 50% by weight, relative to the total weight of the composition.
